(19) United States
(12) Patent Application Publication
(10) Pub. No.: US 2025/0275727 A1
(43) Pub. Date: Sep. 4, 2025

(54) METHOD FOR TRANSMITTING DATA FROM A ROTATING PORTION OF A MEDICAL IMAGING DEVICE TO A STATIONARY PORTION OF THE MEDICAL IMAGING DEVICE AND MEDICAL IMAGING FACILITY

(71) Applicant: Siemens Healthineers AG, Forchheim (DE)

(72) Inventors: Christoph HOHL, Hahnbach (DE); Michael HOSEMANN, Erlangen (DE); Peter Michael DUEPPENBECKER, Herzogenaurach (DE)

(73) Assignee: Siemens Healthineers AG, Forchheim (DE)

(21) Appl. No.: 19/063,700

(22) Filed: Feb. 26, 2025

(30) Foreign Application Priority Data
Feb. 29, 2024 (EP) 24160420.6

(51) Int. Cl. A61B 6/03; A61B 6/00

(52) U.S. Cl. CPC A61B 6/032; A61B 6/563

(57) ABSTRACT

The invention relates to a method for transmitting data from a rotating portion of a medical imaging device to a stationary portion of the medical imaging device. The method includes transmitting the data via radio transmitters on the rotating portion and radio receivers on the stationary portion, wherein each of a plurality of separate transmission pairs is formed, each of the plurality of separate transmission pairs includes one of the radio transmitters and one of the radio receivers, and radio signals are transmitted from the radio transmitter to the radio receiver in the transmission pairs and a main radiation direction of the radio transmitter relating to the radio signals is tracked to the direction of the radio receiver.

### CROSS-REFERENCE TO RELATED APPLICATION(S)

The present application claims priority under 35 U.S.C. § 119 to European Patent Application No. 24160420.6, filed Feb. 29, 2024, the entire contents of which are incorporated herein by reference.

### FIELD

One or more example embodiments relates to a method for transmitting data from a rotating portion of a medical imaging device to a stationary portion of the medical imaging device, wherein the data transmission takes place via radio transmitters arranged on the rotating portion and radio receivers arranged on the stationary portion.

### RELATED ART

Medical imaging devices, such as computed tomography devices, which are often abbreviated to CT devices, frequently have a rotating portion and a stationary portion. The stationary portion is fixed in relation to an environment of the imaging device. When the imaging device is in a rotational state in which, for example, data relating to an image to be generated via the imaging device is captured, the rotating portion rotates or turns relative to the fixed portion. This rotation takes place about a fixed axis of rotation with respect to the stationary portion, which typically forms a system axis of the imaging device.

In the rotational state, the rotating portion often generates large amounts of data that have to be transmitted to the stationary portion for further processing. This transmission typically has to take place as quickly as possible, for example immediately after the data has been generated. The not inconsiderable amount of data generated often does not allow the rotating portion to store this data temporarily, so that there is no alternative to immediate transmission. This problem will increase in future, in particular since modern imaging methods generate ever larger amounts of data due to higher spatial and energy resolutions.

Data transmission frequently takes place via a slip ring system in which an electrical sliding contact creates a coupling between the rotating section and the stationary section that enables data transmission. However, this concept frequently reaches its limits when the currently required data transmission rate exceeds the maximum data transmission rate that can be realized via the slip ring system. A further disadvantage is that wear effects and/or contamination can impair data transmission via the sliding contact.

US 2016/0256129 A1 describes a possible solution to these problems. In the computed tomography device described there, data is transmitted from a rotating part to a stationary part by radio.

### SUMMARY

One or more example embodiments develops the concept of radio-based data transmission from a rotating portion to a stationary portion in a medical imaging device, such as with regard to a high data transmission rate.

Advantages, features and aspects explained for one of the embodiments are also transferable to the other embodiments, unless they expressly and incompatibly deviate from one another.

### BRIEF DESCRIPTION OF THE DRAWINGS

Further advantages, details and features of the present invention emerge from the exemplary embodiments set out below and the figures. The figures show schematically:

FIG. 1 illustrates a perspective view of a medical imaging facility according to an exemplary embodiment,

FIG. 2 illustrates a schematic view of a medical imaging device of the medical imaging facility in FIG. 1, viewed along a system axis according to a first embodiment,

FIG. 3 illustrates a medical imaging facility according to a first alternative of a second embodiment,

FIG. 4 illustrates a medical imaging facility according to a second alternative of the second embodiment,

FIG. 5 illustrates a medical imaging facility according to a third embodiment, and

FIG. 6 illustrates a longitudinal section through a signal conductor of the medical imaging device in FIG. 5 according to an example embodiment.

### DETAILED DESCRIPTION

According to the first embodiment according to the invention, the problem on which example embodiments are based is solved by a method as described in the introduction in that a plurality of separate transmission pairs in each case comprising one of the radio transmitters and one of the radio receivers is formed, wherein, in the transmission pairs, in each case radio signals are transmitted from the radio transmitter to the radio receiver and a main radiation direction of the radio transmitter relating to the radio signals is tracked to the direction of the radio receiver.

With regard to this embodiment according to the invention, the embodiment is partially based on the concept that pauses in the data transmission are avoided. Thus, from the perspective of the respective radio transmitter arranged on the rotating portion, the radio receiver would inevitably move out of the main radiation direction of this radio transmitter due to the rotation. To avoid this, the main radiation direction of the radio transmitter can be changed relative to the rotating portion so that the apparent change in the position of the radio receiver is compensated by tracking the main radiation direction and the transmission of the radio signals can be maintained thereby. The main radiation direction should be understood to mean a spatial direction toward which the radio signals or the electromagnetic waves forming the radio signals are emitted with the greatest intensity compared to the other spatial directions. In extreme cases, the radio signals are emitted exclusively in the main radiation direction.

Assuming that the main radiation direction was fixed, it would rotate synchronously together with the rotating portion. In relation to the transmission pair, the result of this would be that the transmission of radio signals from the respective radio transmitter to the respective radio receiver would only be possible at moments when the main radiation direction meets the position of the radio receiver. With this transmission pair, the transmission of radio signals would only be possible at discrete time points or within narrow time windows, which would severely limit the data transmission rate. The tracking of the main radiation direction provided according to one or more example embodiments enables these time windows to be enlarged.

The radio transmitters can in each case have an antenna via which the radio signals can be generated. Control signals can be used to generate mobile charge carriers in the antenna and these in turn cause the radio signals to be generated. Herein, the radiation intensity follows a lobe-shaped distribution, wherein the main radiation direction corresponds to a central axis of the lobe or one of the lobes. The control signals can be generated via a control facility connected to the radio transmitters. The control facility can be a component of the medical imaging device. The medical imaging device and the control facility can form a medical imaging facility.

The radio receivers can in each case have an antenna via which the radio signals are detected. Electromagnetic waves or radio signals impinging on the antenna generate mobile charge carriers in the antenna and this in turn enables the detection of these electromagnetic waves or radio signals, in particular via the control facility connected to the radio receivers.

In particular, since their physical embodiments can be similar or even identical and only differ with regard to their actuation, at least one of the radio transmitters can in principle also be used as a receiver for radio signals and vice versa, so that data transmission from the stationary portion to the rotating portion is also enabled, for example for transmitting control data or commands. Thus, in addition to unidirectional data transmission from the rotating portion to the stationary portion, bidirectional data transmission is also conceivable within the scope of the present invention. In addition or alternatively, a slip ring system as described in the introduction can also be provided for this purpose.

The radio transmitters can be fixed, i.e. arranged immovably on the rotating portion. The main radiation direction can be changed mechanically and/or electronically. With regard to the mechanical change, it is conceivable that at least one of the radio transmitters has a fixed portion permanently mounted on the rotating portion and a movable portion connected to the fixed portion via a joint, in particular a pivot joint. An actuator, in particular an electromechanical actuator, which can, for example be actuated via the control facility can effect a movement of the movable portion relative to the fixed portion so that the radio signals generated by the movable portion change their main radiation direction. In this embodiment, a mechanically pivotable antenna is realized.

With regard to the electronic change in the main radiation direction, it is conceivable that at least one of the radio receivers has or is a beamforming antenna. This can realize a phased array antenna comprising a plurality of individually actuatable individual antennas, wherein the actuation of the individual antennas, for example via the control facility, effects the adjustment of a directivity and thus the main radiation direction. In this context, the antenna is often referred to as being electronically pivotable.

Sensors connected to the radio transmitters can be arranged on the rotating portion to capture data relating to an image to be generated via the imaging device. The data transmission from the rotating portion to the stationary portion primarily serves to transmit this data. The sensors can in each case be or comprise at least one detector, for example an X-ray detector with, for example, a scintillator.

It is conceivable that at least one of the radio transmitters and at least one of the sensors are realized as a common module that is arranged on the rotating portion. The module can have a carrier, for example a printed circuit board, on and/or in which the components that realize the radio transmitters and the respective sensor are arranged. The respective radio transmitter is used to transmit the data generated via the sensor of this module.

To realize the data transmission, at least one transmission pair is formed, which comprises one of the radio transmitters and one of the radio receivers. Herein, each of the radio transmitters and each of the radio receivers is assigned to exactly, or at most one transmission pair. Preferably, the number of radio transmitters, the number of radio receivers and the number of transmission pairs are the same, in particular for the entire duration of the data transmission.

A transmission channel via which the radio signals are transmitted is in each case formed for the transmission pairs. Therefore, the maximum number of transmission channels available to realize the data transmission corresponds to the number of transmission pairs. The aforementioned tracking of the main radiation direction can take place for all transmission pairs.

According to one or more example embodiments, it is provided that the transmission pairs are specified at the beginning of the data transmission and/or updated during the data transmission in that the radio transmitter of the respective transmission pair is assigned the radio receiver which currently has the shortest distance to this radio transmitter and/or which can be currently connected to the respective radio transmitter via a direct line of sight. In this embodiment, there is no arbitrary assignment between the radio transmitters and the radio receivers with regard to the specification of the transmission pairs. Instead, a specific assignment rule is used for this purpose. Thus, the transmission pairs are formed in such a way that not only is data transmission in principle possible via them, but that this can also take place with the highest possible transmission quality.

This is, for example, clearly the case for transmission pairs whose partners are as close together as possible. Thus, the intensity or the signal strength of the electromagnetic waves representing the radio signals decreases due to free space attenuation as the length of the transmission path increases, so that the transmission path should be as short as possible in order to realize the highest possible transmission quality. Furthermore, it is advantageous in this respect if the respective radio transmitter and the respective radio receivers can be connected to one another via an immediate and direct line of sight, so that the radio signals are transmitted without reflection and therefore along a completely straight and kink-free transmission path. Although data transmission is in principle also conceivable if the radio signals are reflected on their transmission path, for example on the rotating portion and/or the stationary portion, this would complicate tracking accordingly and potentially lead to an impairment of the transmission quality.

The assignment of the radio transmitters and the radio receivers to the transmission pairs takes place at the start of the data transmission, so that this can also be referred to as an initial assignment. In addition, the rotation-related change in the relative positions between the radio transmitters and the radio receivers means that updates or new assignments of the radio transmitters and the radio receivers to the transmission pairs are required during the further course of the data transmission.

In the course of the updating of the transmission pairs, it is necessary for the main radiation direction of the respective radio transmitter to be changed from the direction of the radio receiver assigned to this radio transmitter before the update to the direction of the radio receiver assigned to this radio transmitter after the update. Thus, in a first step, the transmission of the radio signals from the radio transmitter to the radio receiver of the transmission pair present before the update can be terminated. In the second step, the main radiation direction can be changed, for example pivoted. In the third step, the transmission of the radio signals from the radio transmitter to the radio receiver of the transmission pair present after the update can be started.

It is conceivable that at least one quality parameter relating to the quality of the transmission of the radio signals in the respective transmission pair is ascertained, wherein an update time at which the or an update of the at least one transmission pair takes place is specified on the basis of the at least one quality parameter. According to this embodiment, the establishment for the update or the determination of the update times takes place directly by measurement.

The quality parameter or one of the quality parameters can relate to the intensity or signal strength of the radio signals received via the respective radio receiver. This enables an update criterion to be checked and the respective transmission pair to be updated if this criterion is met. The update criterion can be met if the quality parameter indicates that the quality falls below a possibly fixed specified quality limit value, in particular if the intensity falls below an intensity limit value. In this case, the low quality implies that the distance between the radio transmitter and the radio receiver has become so great, or that there is no longer a direct line of sight between the radio transmitter and the radio receiver so that transmission of the radio signals with sufficient quality is no longer possible and this transmission pair should be updated.

It is conceivable that, in addition or alternatively, at least one rotation parameter describing the rotation of the rotating portion is ascertained, wherein the or an update time at which the or an update of the at least one transmission pair takes place is specified on the basis of the at least one rotation parameter. According to this embodiment, the establishment for the update or the determination of the update times takes place indirectly via an analysis, in particular a computational analysis, of the rotation of the rotating portion.

The rotation parameter can relate to a rotation frequency of the rotation of the rotating portion. The or a or a further rotation parameter can relate to a phase of the rotation of the rotating portion. Thus, for example, control signals for controlling the rotation of the rotating portion can be generated via the control facility and transmitted to the rotating portion or an electric motor connected to the rotating portion. A user input and/or a fixed program sequence can be used as the control basis for specifying the control signals, wherein this control basis is used to ascertain at least one rotation parameter.

The at least one rotation parameter can be used to determine time-dependent relative positions between the radio transmitters and the radio receivers, wherein these values are determined as a basis for establishing the update time or update times. For this purpose, time intervals can be determined during which there is a direct line of sight between the respective radio transmitter and the respective radio receiver, wherein an update is indicated at the end of this time interval.

With regard to the specification of the update time, it is conceivable that both the at least one quality parameter and the at least one rotation parameter are used. Thus, the update time can be determined by calculation using the rotation parameter and verified by measurement using the quality parameter. In particular, it is conceivable that the rotation parameter is also corrected or updated using the quality parameter.

It is conceivable that the update takes place simultaneously for at least two of the transmission pairs, in particular for all transmission pairs. Thus, the respective radio transmitters and the respective radio receivers can be arranged in a symmetrical manner, in particular in a uniform manner, in such a way that the updating occurs simultaneously for the transmission pairs.

In addition to or independently of the aspects set out above in connection with the determination of the update times, it is conceivable that the tracking of the main radiation direction takes place based on the at least one quality parameter and/or the at least one rotation parameter. In other words, the at least one quality parameter and/or the at least one rotation parameter can be used as a control basis for the determination of the update times and/or the tracking of the main radiation direction.

Preferably, during a complete rotation of the rotating portion, each of the radio transmitters forms one of the transmission pairs with each of the radio receivers. Herein, the transmission pairs are updated during the rotation of the rotating portion, and therefore during the data transmission, in such a way that all possible transmission pairs are formed successively, so that as few as possible time windows, in particular no time windows, arise during the data transmission during which no or only reduced data transmission is possible.

According to the second embodiment according to the invention, a problem is solved with a method of the type mentioned in the introduction in that a plurality of separate transmission pairs in each case comprising one of the radio transmitters and one of the radio receivers is formed, wherein in each case radio signals are transmitted from the radio transmitter to the radio receiver in the transmission pairs, wherein in each case at least one item of assignment information is ascertained for the radio signals received via the radio receivers in each case, with said item of assignment information describing the radio transmitter which has generated this radio signal, wherein, in the transmission pairs, in each case radio data received by the radio receiver which was not generated by the radio transmitter of this transmission pair is filtered out via the at least one item of assignment information.

According to this embodiment, the data transmission takes place simultaneously or at the same time via a plurality of transmission channels which are in each case realized by one of the transmission pairs, wherein the maximal possible data transmission rate correlates with the number of realized transmission channels. Thus, in this embodiment, radio signals are generated simultaneously via the radio transmitters. Here, the problem is that in each case the radio receivers capture radio signals from a plurality of radio transmitters. Consequently, filtering is required to ensure that, for each transmission pair, only the radio signals received by the radio receiver that originate from the radio transmitter assigned to the respective receiver are used and reused in the course of the respective transmission channel. For this purpose, in each case the radio signals are assigned the assignment information that is captured together with the respective radio signal and can be used to determine the radio transmitter from which this radio signal originates. With regard to the second embodiment according to the invention, for each of the transmission pairs, the only received radio signals used for the respective transmission channel are those for which it is apparent from the assignment information that they were generated by the radio transmitter of this transmission pair. Thus, a so-called MIMO concept is realized, wherein “MIMO” stands for “multiple-input multiple-output”.

The assignment information or one item of the assignment information can be distance information relating to a distance between the respective radio receiver and the radio transmitter via which the respective radio signal was generated. Thus, the radio transmitters are arranged at different positions on the rotating portion so that the respective distances to the radio receivers also differ. In this embodiment, this circumstance is used for the assignment of the radio signals to the radio transmitters.

The distance information can relate to a signal strength or intensity of the respective radio signal, wherein the distance is described indirectly based on the signal strength. If the transmission from the radio transmitter to the radio receiver takes place without reflection, the transmission path corresponds to the direct line of sight or connecting line between the respective radio transmitter and the respective radio receiver. If reflection occurs with this transmission, the transmission path is correspondingly longer than the connecting line. The above-described quality parameter relating, for example, to the intensity of the radio signals can be used as the distance information.

During the course of the filtering, it can be provided that the current distance between the radio transmitter and the radio receiver is ascertained for each of the transmission pairs, for example based on the rotation parameter. This result can be compared with the distance information, wherein further processing or use of the respective radio signal only takes place if there is a match.

It is conceivable that the assignment information or one item of the assignment information is polarization information relating to the polarization of the respective radio signal. Thus, the radio transmitters can in each case be used to generate radio signals with a polarization specific to the respective radio transmitter. A linear polarization, in particular a horizontal, vertical or diagonal, or circular or elliptical polarization, is conceivable for this purpose. Thus, a measured variable can in each case be determined for the radio signals captured by the radio receivers and used to determine polarization information or to represent the polarization information.

Preferably, the polarizations of the radio signals generated in each case via the radio transmitters are known or stored. During the course of the filtering, the polarization of the radio signals can be ascertained or retrieved for each of the transmission pairs. This polarization can be compared with the polarization information, wherein further processing or use of the respective radio signal only takes place if there is a match.

In principle, it is conceivable that the radio signals generated via the radio transmitters or the electromagnetic waves forming the radio signals have the same frequency or are at least located in the same frequency band, wherein at least one of the aforementioned items of information enables the radio signals to be sufficiently differentiated. Nevertheless, the assignment information or one item of the assignment information can be frequency information relating to a frequency or a frequency band of the respective radio signal. Thus, the radio transmitters can in each case be used to generate radio signals with a frequency specific to the respective radio transmitter. In this respect, specific frequency bands with a bandwidth of 2 GHZ, for example, are also conceivable. Accordingly, it is provided that a measured variable is in each case determined for the radio signals captured by the radio receivers and used to determine the frequency information or to represent the frequency information.

Preferably, the frequencies of the radio signals generated in each case via the radio transmitters are known or stored. During the course of the filtering, it can be provided that the frequency or frequency band of the respective radio signals is ascertained or retrieved for each of the transmission pairs. This frequency or this frequency band can be compared with the frequency information, wherein further processing or use of the respective radio signal only takes place if there is a match.

With regard to the options explained, it is provided that only one item of this assignment information is ascertained and evaluated. Preferably, however, at least two, particularly preferably three, items of this assignment information are ascertained and evaluated. Thus, the distance information can be provided as the first assignment information, the polarization information can be provided as the second assignment information and the frequency information can be provided as the third assignment information.

If the assignment information or one item of the assignment information is the frequency information, it can be provided that the radio transmitters are in each case assigned to one of a plurality of radio transmitter groups, wherein the radio transmitters in one of the radio transmitter groups in each case generate radio signals in the same frequency range, wherein the radio transmitters in different radio transmitter groups in each case generate radio signals in different frequency ranges, wherein the radio transmitters in the radio transmitter groups are in each case arranged in groups and at a distance to the radio transmitters in the at least one other radio transmitter group on the rotating portion. If the radio signals are in the same frequency range, they have the same frequency or are in the same frequency band. The radio transmitters in the radio transmitter groups are in each case arranged in clusters next to one another. While the radio transmitters in the radio transmitter groups are in each case in particular arranged directly next to one another or adjacent to one another, there is a greater distance between the radio transmitter groups in this respect.

In this embodiment, the frequency information enables prefiltering with regard to the radio transmitter groups before the final filtering of the radio signals. Due to the local proximity of the radio transmitters in the respective radio transmitter groups, it can be assumed that the radio signals generated by these radio transmitters are mainly or predominantly radio signals received by radio receivers that also belong to a radio receiver group and are arranged in local proximity to one another on the stationary portion. Specifically, the radio receivers in the radio receiver groups can therefore in each case also be arranged in groups and at a distance to the radio receivers in the at least one other radio receiver group on the stationary portion. The radio receivers in the radio receiver group are in each case arranged in clusters next to one another. While the radio receivers in the radio receiver group are in each case arranged directly next to one another or adjacent to one another, there is a greater distance between the radio receiver groups in this respect.

The number of radio transmitter groups preferably corresponds to the number of radio receiver groups. The assignments of the radio transmitters or radio receivers to the radio transmitter groups or radio receiver groups can be fixed, in particular if the components of the radio transmitter groups or radio receiver groups are arranged in local proximity to one another. The transmission pairs can be formed in such a way that the radio transmitters in one of the radio transmitter groups are only assigned to the radio receivers of one of the radio receiver groups and vice versa. Therefore, in this embodiment, group pairs are formed, wherein the radio transmitters in the radio transmitter group of this group pair and the radio receivers in the radio receiver group of this group pair in each case form transmission pairs.

Preferably, the frequencies of the radio signals generated in each case via the radio transmitters in one of the radio transmitter groups are known or stored. In the course of the prefiltering, it can be provided that the frequency or frequency band of the respective radio signals is known for each of the group pairs. This frequency or this frequency band can be compared with the frequency information, wherein further processing of the respective radio signal during the course of the filtering only takes place if there is a match. The selection effect realized during the course of the prefiltering causes a further increase in the maximum possible data transmission rate by a certain factor without this further complicating the above-described filtering. This factor corresponds to the number of group pairs provided.

Preferably, it is provided in the second embodiment according to the invention that the radio transmitters in each case have at least one main radiation direction of the radio signals which is fixed with respect to the rotating portion. In contrast to the first embodiment according to the invention, there is no change in the main radiation direction with respect to the rotating portion. Instead, the main radiation direction rotates together and simultaneously with the rotating portion. The main radiation direction of the radio receivers can in each case cover a plurality of radio receivers or preferably all radio receivers in succession. In this case, the transmission pairs are updated such that the radio transmitters are in each case assigned the radio receiver that is currently arranged in the region of the main radiation direction. The rotation parameter can also be used in this respect. With regard to the above-described filtering, the presence of the main radiation direction is advantageous, because, due to this directional characteristic, a further filter effect is realized almost automatically, without this requiring specific evaluation steps. Thus, in the case of typical directional characteristics, the number of radio transmitters whose radio signals are received simultaneously via one of the radio receivers is limited, thereby significantly simplifying the above-described filtering.

The radio transmitters can in each case comprise or be at least one patch antenna via which the radio signals can be generated. The patch antenna typically has a directional characteristic and therefore a main radiation direction. The antenna, in particular the patch antenna, can be integrated on a printed circuit board of the radio transmitter. The patch antenna can, for example, be realized as a metal surface, in particular a rectangular metal surface, which is arranged on the printed circuit board.

It is conceivable that at least one of the radio receivers has a main receiving direction that relates to a spatial direction that is sensitive with respect to the reception of the radio signals. This means that the radio receiver can only receive radio signals that impinge on the radio receivers from the direction of the main receiving direction.

Preferably, the main radiation direction of at least one of the radio transmitters and the main receiving direction of at least one of the radio receivers point along and/or against a direction of rotation relating to the rotation of the rotating portion. The main radiation direction can run along a tangential direction of the rotating portion, which is circular in cross section, or deviate herefrom by a maximum of a small angle, for example by a maximum of 20°. The main receiving direction can run along a tangential direction of a receiving portion with a circular cross section of the stationary portion or deviate herefrom by a maximum of a small angle, for example by a maximum of 20°.

Preferably, the radio transmitters are in each case assigned to a forward-directed radio transmitter set or backward-directed radio transmitter set. It is provided for the radio transmitters with the forward-directed radio transmitter set that their main radiation directions point along the direction of rotation. It is provided for the radio transmitters with the backward-directed radio transmitter set that their main radiation directions point against the direction of rotation. Particularly preferably, the radio receivers are also in each case assigned to a forward-directed radio receiver set or a backward-directed radio receiver set. It is provided for the radio receivers with the forward-directed radio receiver set that their main receiving directions point along the direction of rotation. It is provided for the radio receivers with the backward-directed radio receiver set that their main receiving directions point against the direction of rotation. The transmission pairs are formed in such a way that they in each case comprise either one of the radio transmitters with the forward-directed radio transmitter set and one of the radio receivers with the backward-directed radio receiver set or one of the radio transmitters with the backward-directed radio transmitter set and one of the radio receivers with the forward-directed radio receiver set. In the other cases, the formation of the transmission channel is not possible because the main radiation direction and the main receiving direction substantially point in the same direction and this excludes the transmission of the radio signals. This additional selection effect causes a further increase, namely a doubling, of the maximum possible data transmission rate, without this further complicating the above-described filtering.

According to the third embodiment according to the invention the problem is solved with a method of the type mentioned in the introduction in that radio signals transmitted from the radio transmitters to the radio receivers are coupled into at least one signal conductor leading to the radio receivers and consisting of a dielectric material, wherein the signal conductor has a permittivity that changes transversely to a direction of extension.

With regard to the third embodiment according to the invention, due to the changing permittivity, a direction of propagation of the radio signals in the signal conductor is deflected toward the direction of extension of the signal conductor until these two directions run parallel to one another. The radio signals can be coupled into the signal conductor. Herein, the angle between the signal conductor or its direction of extension and the direction of propagation of the radio signals can be greater than 0° and less than 90°, preferably less than 45°.

If this permittivity did not change, the radio signals would run through the signal conductor along the transverse direction and emerge again from the signal conductor on the side opposite the coupling side. Instead, the change in permittivity, in particular an increase in permittivity, causes the aforementioned alignment of the direction of propagation of the radio signals with the direction of extension, so that the radio signals are guided along the direction of extension within the signal conductor. In other words, the permittivity that changes transversely or perpendicular to the direction of extension leads to a quasi-continuous refraction of the electromagnetic wave. A direction of propagation of the radio signals that runs at an angle to the direction of extension, which is present immediately after coupling into the signal conductor, is deflected in the direction of the direction of extension due to the changing permittivity until the direction of propagation corresponds to the direction of extension.

Therefore, part of the transmission path along which the radio signals travel during transmission from the radio transmitter to the radio receiver runs through the signal conductor thus enabling the radio signals to be routed to the radio receiver in a targeted manner. In particular, this solves a problem that is often relevant when multiple reflections occur, namely that interference in the radio signals leads to an impairment of the data transmission.

The permittivity, which is often also referred to as dielectric conductivity, dielectricity or dielectric function and is frequently abbreviated with the symbol ε, indicates a measure of the polarization capacity of the material. Preferably, the permittivity changes continuously transversely, in particular perpendicularly, to the direction of extension, i.e. without the course of the permittivity exhibiting jumps along this transverse direction. The range of values for the permittivity is selected in such a way that the attenuation of the radio signals is as low as possible for the selected frequency range. The signal conductor can be made of plastic. In this respect, polystyrene (PS) and/or polyethylene (PE) and/or polypropylene (PP) and/or polytetrafluorethylene (PTFE) are conceivable.

The signal conductor can have a rectangular shape when viewed in cross section, i.e. perpendicular to the direction of extension. The direction of extension can run along the central fiber of the signal conductor, i.e. comprise the midpoints of the cross-sectional planes. The direction of extension can also be referred to as a longitudinal direction of the signal conductor. The signal conductor, i.e. its direction of extension, preferably extends along the circumference of the receiving portion of the stationary portion. The signal conductor or the direction of extension therefore forms a closed, preferably circular, ring. This ring can surround the system axis concentrically. The radio signals are coupled into the signal conductor laterally and radially on the inside when they impinge thereupon. The permittivity increases radially outward. With regard to the radial direction, the gradient of permittivity is therefore positive, in particular constant.

The radio receivers can be arranged within the signal conductor. In other words, the radio receivers can be embedded in the dielectric material. Therefore, the radio signals impinge on the respective radio receiver after being coupled into the signal conductor, without the radio signals having to be decoupled from the signal conductor beforehand.

The signal conductor can have a changing geometric structure, wherein the changing permittivity is realized via the changing geometric structure. Thus, the geometric structure can form a pattern in which a unit cell or elementary cell repeats cyclically. It is conceivable that the structure is honeycomb-shaped, i.e. forms a hexagonal lattice structure. The size of the unit cell or elementary cell can change along the transverse direction of the signal conductor in order to realize the changing permittivity. To realize the changing geometric structure, it is conceivable that the signal conductor is produced via a 3D printer.

The aspects explained in the context of the second embodiment are in particular equally applicable to the third embodiment. This applies in particular to the aspects explained above with regard to the main radiation directions and main receiving directions pointing along or pointing against the direction of rotation. Thus, the radio signals can be guided through the ring-shaped signal conductor along two opposite directions of propagation.

With regard to all three embodiments, it is conceivable that the stationary portion has the or a cylindrical receiving region in which the rotating portion is arranged. The radio transmitters can be arranged along a circumference of the rotating portion. The radio receivers can be arranged along a circumference of the receiving region. Preferably, the receiving portion is a hollow free space, in particular a circular-cylindrical free space, which is bounded by a housing of the stationary portion. Accordingly, the rotating portion is likewise preferably embodied as cylindrical, in particular as circular-cylindrical, and is optionally connected to the stationary portion via a ring bearing. The rotating portion can be rotated via a drive means, for example an electric motor, which is actuated via the control facility. The frequency of rotation can typically be up to several hundred revolutions a minute.

The radio transmitters and/or the radio receivers can be arranged equidistantly along the circumference. For example, up to 50 radio transmitters and up to 50 radio receivers can be provided. The radio transmitters can be arranged along an outer circumference of the rotating portion. The radio receivers can be arranged along an inner circumference of the receiving portion. The radio transmitters and the radio receivers are preferably arranged along the lines of two concentric circles that describe the respective circumference.

In addition to the method according to one or more example embodiments, one or more example embodiments also relates to a medical imaging facility. This comprises a medical imaging device, in particular a computed tomography device, having a rotating portion and a stationary portion, wherein a plurality of radio transmitters is arranged on the rotating portion and a plurality of radio receivers is arranged on the stationary portion, wherein the radio transmitters and the radio receivers enable data transmission from the rotating portion to the stationary portion.

According to the first embodiment according to the invention, the problem is solved with a medical imaging facility as described above in that a control facility is provided that is configured to generate control signals for controlling the operation of the radio transmitters and the radio receivers in such a way that at least one transmission pair is formed, which, on the one hand, comprises the radio transmitters or one of the radio transmitters and, on the other, comprises the radio receiver or one of the radio receivers, wherein, in the transmission pair or at least one of the transmission pairs, a wireless transmission channel for transmitting radio signals from the radio transmitter to the radio receiver is formed and a main radiation direction of the radio transmitter relating to the radio signals is tracked to the direction of the radio receiver.

According to the second embodiment according to the invention, the is solved with a medical imaging facility as described above in that a control facility is provided that is configured to generate control signals for controlling the operation of the radio transmitters and the radio receivers in such a way that a plurality of separate transmission pairs in each case comprising one of the radio transmitters and one of the radio receivers is formed, wherein in the transmission pairs in each case radio signals are transmitted from the radio transmitter to the radio receiver, wherein in each case at least one item of assignment information is ascertained for the radio signals received via the radio receivers, with said item of assignment information describing the radio transmitter which has generated this radio signal, wherein in the transmission pairs in each case radio data received by the radio receiver which was not generated by the radio transmitter of this transmission pair is filtered out via the at least one item of assignment information.

According to the third embodiment according to the invention, the problem is solved with a medical imaging facility as described above in that radio signals transmitted from the radio transmitters to the radio receivers can be coupled into at least one signal conductor leading to the radio receivers and consisting of a dielectric material, wherein the signal conductor has a permittivity that changes transversely to a direction of extension.

It also applies with respect to the medical imaging facility according to one or more example embodiments that the advantages, features and aspects explained for one of the embodiments are also applicable to the other embodiments provided that the respective embodiments do not incompatibly deviate from one another in this respect. Moreover, all advantages, features and aspects explained in connection with the method according to one or more example embodiments are equally transferable to the medical imaging facility according to one or more example embodiments and vice versa.

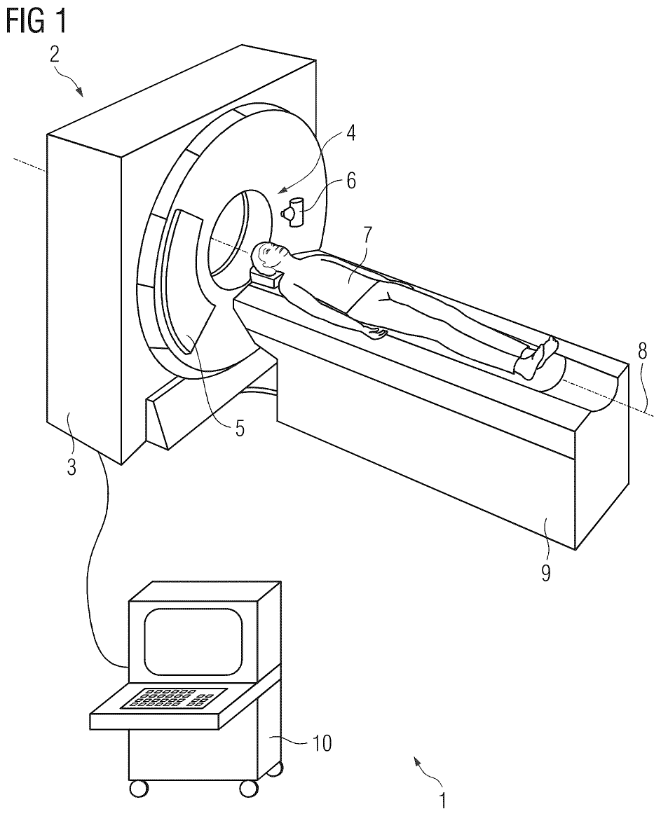

FIG. 1 shows a medical imaging facility 1 according to one or more example embodiments with a medical imaging device 2; in the present case a computed tomography device. The imaging device 2 comprises a stationary portion 3 and a rotating portion 4, wherein the rotating portion 4 forms the gantry of the computed tomography device. The rotating portion 4 is provided with an emitter-detector system consisting of a modular X-ray detector 5 and the opposite X-ray source 6. Alternatively, a plurality of emitter-detector systems can also be provided on the rotating portion 4.

A housing of the stationary portion 3 bounds a circular-cylindrical receiving portion in which the rotating portion 4 is arranged and in which an X-ray measuring field of the active emitter-detector system is formed during operation. For the measurement, a patient 7, who is located on a patient bench 9 that can be moved in the direction of a system axis 8, is pushed continuously or step-by-step through the measuring field while the rotating portion 4 rotates about the system axis 8. Herein, the attenuation of the X-rays emitted by the X-ray tube through the patient 7 is measured pixel-by-pixel via sensors, wherein preferably direct-converting sensor materials, for example a scintillator, are used and the incident X-ray photons are counted in an energy-resolved manner.

The detector data ascertained in this way is transmitted from the rotating portion 4 to the stationary portion 3 in the course of a wireless data transmission. The relevant control and also the evaluation of the received measured data for the realization of the medical imaging takes place via a control facility 10 of the medical imaging facility 1, namely with the aid of software implemented there and executed during operation. In addition, the control facility 10 is configured to control the operation of radio transmitters 11, 13, 17, 18 and radio receivers 12, 15, 19, 20. Although the following explains unidirectional data transmission from the rotating portion 4 to the stationary portion 3, it is in principle also conceivable that data is transmitted in the opposite direction and that bidirectional communication takes place, for example for control purposes relating to the detectors. In addition or alternatively, data transmission from the stationary portion 3 to the rotating portion 4 is also conceivable via a slip ring system in which an electrical sliding contact establishes a coupling between the rotating portion 4 and the stationary portion 3.

With regard to the rotation of the rotating portion 4, the control facility 10 is configured to actuate an electric motor, which is not shown in the figures and is coupled to the rotating portion 4. The electric motor is coupled to the rotating portion 4 in such a way it can be used to rotate the rotating portion 4 according to predefined rotation parameters. The rotation parameters that describe the rotation of the rotating portion 4 are, for example, specified via user input and/or a fixed program sequence stored by the control facility 10. One of the rotation parameters relates to the rotation frequency of the rotating portion 4. A further rotation parameter relates to a phase of this rotation, i.e. the position of the rotating portion 4 when the rotation passes through zero.

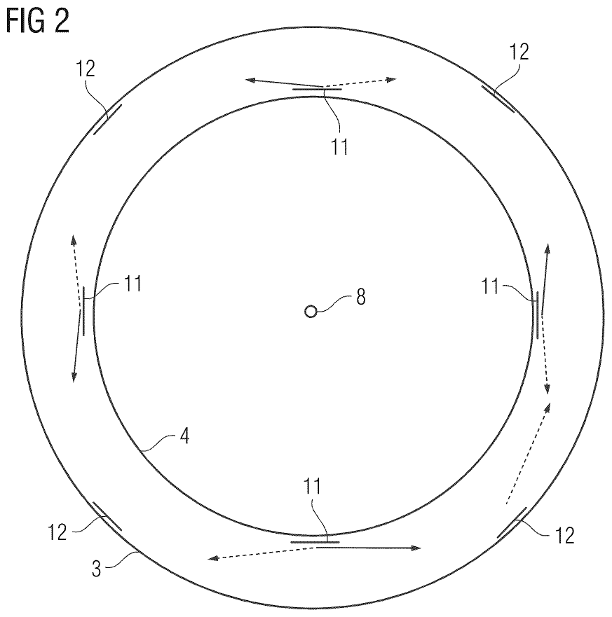

The following explains a first variant with reference to FIG. 2, specifically relating to the medical imaging facility 1 and the method according to one or more example embodiments. FIG. 2 shows a view of the imaging device 2 looking along the system axis 8. Like the other components shown in this figure, the portions 3, 4 are only indicated very schematically.

To realize the data transmission, radio transmitters 11 arranged on the rotating portion 4 and radio receivers 12 arranged on the stationary portion 3 are provided, namely, by way of example, four in each case. The radio transmitters 11 are arranged equidistantly along an outer circumference of the cylindrical rotating portion 4. The radio receivers 12 are arranged equidistantly along an inner circumference of the cylindrical receiving portion. The radio transmitters 11 and the radio receivers 12 are in each case arranged along an imaginary circular line of two concentric circles, through the center of which the system axis 8 runs.

With reference to FIG. 2, it is assumed that the rotating portion 4 rotates along a direction of rotation, in the present case clockwise, by way of example. To define spatial directions that in FIG. 2 extend radially away from the system axis 8, it is assumed that the direction vertically upward, corresponding to a twelve o'clock position, is assigned a value of 0° and 360°. Starting from the 0° position, an angle specification of up to 360° relates to rotation along the clockwise direction. Thus, an angle specification of 90° corresponds to a three o'clock position, an angle specification of 180° corresponds to a six o'clock position and an angle specification of 270° corresponds to a nine o'clock position.

In FIG. 2, the radio transmitters 11 are located at 0°, 90°, 180° and 270°, wherein these values increase accordingly during the rotation of the rotating portion 4. The known rotation parameters are used to determine the positions of the radio transmitters 11 as a function of time. The positions of the radio receivers 12 are fixed and are assumed to be known. In FIG. 2, the radio receivers 12 are constantly located at 45°, 135°, 225° and 315°. The relative positions between the radio transmitters 11 and the radio receivers 12 are determined from this, also as a function of time.

Depending on the relative positions between the radio transmitters 11 and the radio receivers 12, four transmission pairs are formed, in each case comprising one of the radio transmitters 11 and one of the radio receivers 12. For this purpose, each of the radio transmitters 11 is assigned exactly the radio receiver 12 that currently has the shortest distance to this radio transmitter 11 with respect to all radio receivers 12. The transmission pairs in each case form a wireless transmission channel for transmitting radio signals from the respective radio transmitter 11 to the respective radio receiver 12. Data transmission takes place via these transmission channels.

Since the relative distances between the components of the transmission pairs are constantly changing, it is necessary for the transmission pairs to be constantly updated in order to maintain data transmission during the rotation of the rotating portion 4. The update times at which a reassignment of the transmission pairs is necessary and carried out are determined using the rotation parameters. With reference to the radio transmitter 11 arranged at 0° in FIG. 2, it is clear that, at the time shown, it is located between two radio receivers 12, namely between the radio receiver 12 at the top right at 45° and the radio receiver 12 at the top left at 315°. Immediately before the time shown in FIG. 2, the radio receiver 12 with the shortest distance to this radio transmitter 11 is the radio receiver 12 at 315°. The corresponding transmission of radio signals is indicated by the solid arrow in FIG. 2. Immediately after the time shown in FIG. 2, the radio receiver 12 with the shortest distance to this radio transmitter 11 is the radio receiver 12 at 45°. The corresponding transmission of radio signals is indicated via the dashed arrow in FIG. 2. The transmission pair with the radio transmitter 11 at 0° is therefore updated at the time shown in FIG. 2 which is consequently one of the update times. This applies analogously to the three further transmission pairs.

In addition to the rotation parameters, a quality parameter is determined that relates to an intensity, and therefore the quality, of the radio signals received via the respective radio receiver 12. Thus, the intensity or signal strength of the radio signals depends on the distance between the radio transmitter 11 and the radio receiver 12 and on whether there is currently a direct line of sight between the radio transmitter 11 and the radio receiver 12. Immediately before the time shown in FIG. 2, there is a direct line of sight for the transmission pair comprising the radio transmitter 11 at 0° and the radio receiver 12 at 315°. When the position shown in FIG. 2 is reached, this line of sight is interrupted by the rotating portion 4, wherein a direct line of sight is created for the transmission pair comprising the radio transmitter 11 at 0° and the radio receiver 12 at 54°. This is reflected accordingly in the quality parameter in which a sudden drop in intensity occurs with respect to the radio transmitter 11 at 0° and the radio receiver 12 at 315° and a sudden increase in intensity occurs with respect to the radio transmitter 11 at 0° and the radio receiver 12 at 45°. Although, according to one or more example embodiments, the quality parameter can be used instead of the rotation parameter to determine the update times, in the present case, all parameters are used for this purpose. Thus, in the present case, the quality parameter is used to verify the update times determined using the rotation parameters. In addition, the rotation parameter relating to the phase is updated or corrected using the quality parameter, for example in order to avoid an ever-increasing deviation of the phase over time.

A further aspect of the exemplary embodiment shown in FIG. 2 relates to a main radiation direction of the radio transmitters 11. The main radiation direction denotes the spatial direction emanating from the respective radio transmitter 11 along which the intensity of the electromagnetic waves generated via this radio transmitter 11 and forming the radio signals has a maximum. Thus, it is provided in this respect that the main radiation direction is tracked in the direction of the radio receiver 12 of the respective transmission pair. Moreover, the main radiation direction is changed at the update time in such a way that it is pivoted from the position of the radio receiver 12 of the transmission pair before the update to the position of the radio receiver 12 of the transmission pair after the update.

The variable main radiation direction is realized by the fact that the radio transmitters 11 in each case comprise an electronically pivotable antenna, namely a beamforming antenna, which is a phased array antenna with between 4 and 64 antenna components. The main radiation direction can be changed and adjusted via electronic actuation of these antenna components by the control facility 10. In the present case, the tracking of the main radiation direction is controlled via the rotation parameters and the quality parameter.

In this embodiment, multi-gigabit transmission is realized, wherein, by way of example, the frequency range used is the ISM band at 60 GHz. Four transmission channels with a transmission bandwidth of 2 GHz are used, resulting in a total data rate of, for example, 25 Gb/s. The specific number of radio transmitters 11 and radio receivers 12 should be understood as being only by way of example, so that, for example, if a data rate of 200 Gb/s is required, eight separate transmission channels and therefore eight radio transmitters 11 and radio receivers 12 can be provided in each case.

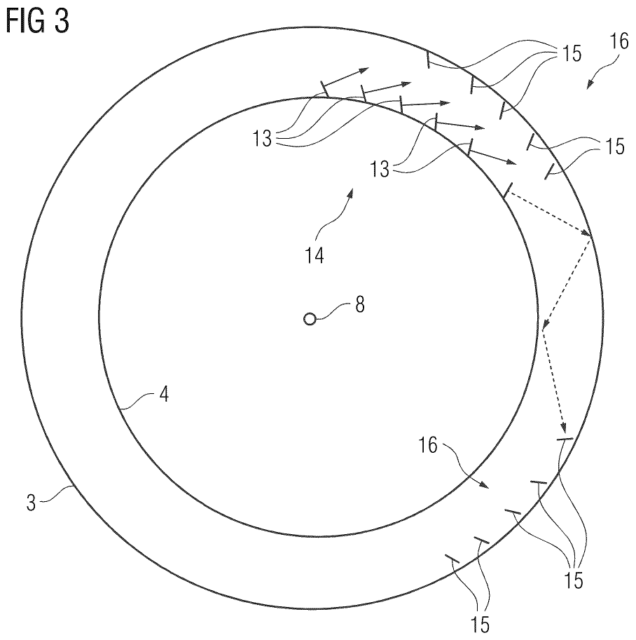

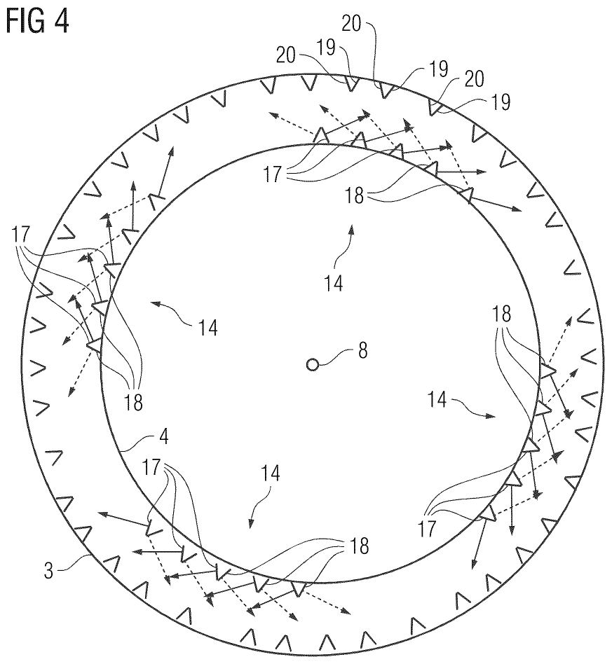

The following explains a second variant with reference to FIG. 3 and FIG. 4. Herein, FIG. 3 relates to a first alternative of the second variant and FIG. 4 relates to a second alternative of the second variant. Reference is first made to FIG. 3 which shows the same view of the imaging device 2 as that shown in FIG. 2. In the exemplary embodiment shown in FIG. 3, a so-called MIMO concept is realized in the medical imaging facility 1.

Thus, the radio transmitters 13 arranged on the rotating portion 4 are assigned to four radio transmitter groups 14, which in each case comprise a plurality of radio transmitters 13 arranged in groups along the circumference of the rotating portion 4. The radio transmitters 13 in the respective radio transmitter group 14 are in each case arranged directly adjacent to one another, wherein, in this respect, there is a greater distance to the radio transmitters 13 in the other radio transmitter groups 14. Consequently, the radio transmitters 13 in each of the radio transmitter groups 14 are arranged in clusters next to one another and at a distance to the radio transmitters 13 in the other radio transmitter groups 14. For the sake of clarity, FIG. 3 only shows one of the radio transmitter groups 14, namely the one provided in the range between 0° and 60°. The other radio transmitter groups are arranged in the ranges between 90° and 150°, between 180° and 240° and between 270° and 330°. Similarly, the radio receivers 15 are also in each case assigned to one of four radio receiver groups 16, wherein FIG. 3 depicts two of the radio receiver groups 16.

In contrast to the exemplary embodiment explained with reference to FIG. 2, in the exemplary embodiment shown in FIG. 3, it is provided that the radio transmitters 13 have a main radiation direction that is fixed with respect to the rotating portion 4. Thus, for this purpose, the radio transmitters 13 in each case have a patch antenna via which the radio signals are radiated in a directed manner. In FIG. 3, the main radiation direction are indicated by the arrows, wherein it can be seen that transmission of the radio signals from the radio transmitters 13 to the radio receivers 15 is also conceivable via reflections on the stationary portion 3 and the rotating portion 4.

Similarly to the main radiation direction of the radio transmitters 13, it is also provided that the radio receivers 15 in each case have a main receiving direction relating to a spatial direction that is sensitive with respect to the reception of the radio signals. In the exemplary embodiment shown in FIG. 3, it can be seen that the main radiation direction points along the direction of rotation relating to the rotation of the rotating portion 4. This means that the main radiation direction, which points along the arrows shown in FIG. 3, substantially points along the tangential direction of the rotating portion 4. The main receiving directions of the radio receivers 15 are accordingly aligned in opposite directions.

With regard to data transmission, it is provided that a plurality of transmission pairs is formed in each case comprising one of the radio transmitters 13 and one of the radio receivers 15. Apart from the explanation given below, the explanations of the transmission pairs given in the context of the first variant also apply to the transmission pairs in the second variant. One difference is that, due to the fixed main radiation directions of the radio transmitters 13 with respect to the rotating portion 4 and the main receiving directions of the radio receivers 15 with respect to the stationary portion 3, data transmission can only take place in those moments or time windows when the fixed main radiation direction currently covers the position of the radio receiver 15 with the opposite main receiving direction in this respect. In relation to FIG. 3, this is the case for the radio transmitters 13 in the radio transmitter group 14 shown at the top right and the radio receivers in the radio receiver group 16 shown at the top right. The main radiation directions and the main receiving directions substantially coincide directionally in such a way that the radio signals generated by the radio transmitters 13 in this radio transmitter group 14 are received by the radio receivers 15 in this radio receiver group 16.

One problem here is the circumstance that each of the radio receivers 15 in the radio receiver group 16 at the top right in FIG. 3 receives the radio signals of all or at least some of the radio transmitters 13 in the radio receiver group 14 shown in this figure. A further problem in this respect is the circumstance that the radio signals in further radio transmitter groups 14 not shown in FIG. 3 are also received via the radio receiver group 14 shown. Although there are no direct lines of sight, this is due to the circumstance that these radio signals reach this radio receiver group 14 via reflections or multiple reflections. A conceivable transmission path for one of these radio signals is shown with respect to the radio transmitter group 14 in relation to the radio transmitter 13 shown in FIG. 3 that is currently arranged furthest to the right.

Due to the circumstance explained above that each of the radio receivers 15 not only captures the radio signals of the radio transmitter 13 currently assigned in the context of the assignment of the transmission pairs, but also those of a plurality of radio transmitters 13, it is necessary to filter the radio signals in this respect. This ensures that, for each of the radio receivers 15, the only radio signals that are further processed are those that were generated by the radio transmitter 13 assigned to the corresponding transmission pair. In relation to FIG. 3, it is provided in the current state of the two groups 14, 16 shown at the top right that the first radio transmitter 13 from the left and the first radio receiver 15 from the left, the second radio transmitter 13 from the left and the second radio receiver 15 from the left and so on in each case form a transmission pair. The specific specification of the transmission pairs is based on the rotation parameters also captured in the context of this alternative, so that the main radiation direction of the respective radio transmitter 13 and the main receiving direction of the respective radio receiver 15 are at least substantially collinear.

To enable filtering of the radio signals, it is provided that a plurality of items of assignment information is in each case ascertained for each of the transmitted radio signals and can be used as the basis for ascertaining the radio transmitter 13 that generated this radio signal. This procedure allows the radio signals that do not originate from the radio transmitter assigned to the respective transmission pair to be discarded for each of the radio receivers 15.

One item of the assignment information provided is frequency information relating to a frequency of the electromagnetic waves forming the respective radio signal. Thus, the radio signals generated by different radio transmitters 13 can have different frequencies. The radio receiver 15 can measure the respective frequency, wherein the corresponding result is the frequency information.

In the exemplary embodiment shown in FIG. 3, a separate MIMO system is realized via each of the radio receiver groups 14. This means that the radio transmitters 13 in one of the radio transmitter groups 14 in each case generate radio signals in the same frequency range, wherein the radio transmitters 13 in different radio transmitter groups 14 in each case generate radio signals in different frequency ranges. This enables the radio signals to be prefiltered. Thus, group pairs comprising one of the radio transmitter groups 14 and one of the radio receiver groups 16 are formed. It is provided in the respective group pair that the radio transmitters 13 in the respective radio transmitter group 14 and the radio receivers 15 in the respective radio receiver group 16 in each case form transmission pairs. In the present case, the two groups 14, 16 shown at the top right in FIG. 3 form one of the group pairs.

The control facility 10 stores the frequencies of the radio signals generated via the radio transmitters in one of the radio transmitter groups 14 in each case. In the context of the prefiltering, it is provided that this known frequency or this known frequency band is compared with the frequency information of one of the radio receivers 15 in the radio receiver group 16 captured in each case, wherein further processing of the respective radio signal in the context of the filtering only takes place if there is a match.

This applies to each of the four group pairs, so that a quadruple MIMO system is realized overall. In this system, it is provided that each of the radio transmitters 13 operates on a bandwidth of 2 GHz and using a quadrature amplitude modulation of 16. The separation of these individual MIMO systems is enabled by the above-described filtering with regard to the frequencies. The radio transmitters 13 in one of the sub-systems or one of the radio receiver groups 14 operate on a common frequency, wherein the frequencies of the four sub-systems or radio receiver groups 14 differ from one another. In this respect, frequencies of 60 GHZ, 62 GHZ, 64 GHz and 66 GHz are conceivable.

One item of the assignment information provided is distance information relating to a distance between the respective radio receiver 15 and the radio transmitter 13 which generated this radio signal. Thus, the known rotation parameters can be used to calculate the relative distance between the radio transmitter 13 and the radio receiver 15 for each of the transmission pairs, wherein a signal strength to be expected for the radio signals generated by the radio transmitter 13 in this transmission pair on reception via the radio receiver 15 in the respective transmission pair is determined on the basis of free-space attenuation and possibly taking into account the directional characteristic. This result is compared with the distance information, wherein only the radio signals for which the values match are further processed and not discarded. On the one hand, the distance information is extremely expedient with regard to the assignment of the radio signals to the respective radio transmitter groups 14 or the prefiltering, since if, as in the present case, a frequency of, for example, 60 GHz is used for the radio signals, there is high free-space attenuation, and this realizes a corresponding selectivity with regard to the distance information. On the other hand, this selectivity is also high enough so that the distance information makes it possible to differentiate between the individual radio transmitters 13 within one of the radio transmitter groups 14 and therefore enables final filtering.

Nevertheless, polarization information relating to the polarization of the respective radio signal is also provided as an item of the assignment information. Thus, the electromagnetic waves forming the radio signals, which are generated by different radio transmitters 13, have different polarizations, for example horizontal, vertical, diagonal, circular or elliptical polarization. Accordingly, for this purpose, the antennas of the radio transmitters 13 generate electromagnetic waves with different polarization properties. The radio receiver 15 can measure the respective polarization, wherein the result is the polarization information. In addition, the polarization of the generated radio signals is known for each radio transmitter 13. The result for the polarization information can be compared with this, wherein only the radio signals for which the polarization information matches the polarization of the radio transmitter 13 in the respective transmission pair are further processed and not discarded.

The following explains a second alternative of the second variant with reference to FIG. 4. FIG. 4 shows the same view of the imaging device 2 as that shown in FIG. 2 and FIG. 3. Unless explicitly stated otherwise, the aspects explained with respect with reference to FIG. 3 apply equally to the exemplary embodiment shown in FIG. 4. In contrast to FIG. 3, all the radio transmitters 17, 18 provided and all the radio receivers 19, 20 provided are shown in FIG. 4. While the radio receivers 19, 20 are arranged evenly distributed along the circumference of the stationary portion 3, the four radio transmitter groups 14 explained above are provided with regard to the radio transmitters 17, 18. The radio receiver groups 16 are not assigned radio receivers 19, 20 arranged in clusters in each case. Instead, the radio receiver groups 16 are determined on the basis of the current positions of the radio receivers 17, 18 relative to the radio receivers 118, 20, which are known on the basis of the rotation parameters. Thus, in each case the radio receivers 19, 20 captured by or impinged upon by the main radiation directions of the radio transmitters 17, 18 of one of the radio transmitter groups 14, 15 are combined to form a radio receiver group. These two groups 14, 16 are combined to form a group pair. Therefore, each of the radio receiver groups 16 virtually migrates along the circumference of the stationary portion 3 together with the rotation of the rotating portion 4 and therefore of the associated radio transmitter group 4.

In addition, it is provided in the exemplary embodiment shown in FIG. 4 that the radio transmitters 17 are assigned to a forward-directed radio transmitter set and the radio transmitters 18 are assigned to a backward-directed radio transmitter set. In the case of the radio transmitters 17, the main radiation directions point along the direction of rotation and in the case of the radio transmitters 18 they point against the direction of rotation. These directions are indicated via solid arrows in the case of the radio transmitters 17 and via dashed arrows in the case of the radio transmitters 18.

Furthermore, the radio receivers 19 are assigned to a forward-directed radio receiver set and the radio receivers 20 to a backward-directed radio receiver set. In the case of the radio receivers 19, the main receiving directions point along the direction of rotation and, in the case of the radio receivers 20, they point against the direction of rotation. Due to these directional characteristics of the radio transmitters 17, 18 and the radio receivers 19, 20, transmission pairs can in principle only be formed between the radio transmitters 17 in the forward-directed radio transmitter set and the radio receivers 20 in the backward-directed radio receiver set and vice versa.

Due to the fact that, in the exemplary embodiment shown in FIG. 4, a plurality of radio transmitter groups 14 is provided, similarly to the aspects explained with reference to FIG. 3, a multiple MIMO system is realized. Moreover, due to the diametrically opposed directional characteristics or the further division of the radio transmitters 17, 18 and radio receivers 19, 20 into forward-directed and backward-directed groups, a selection effect is realized in relation to the receivability of the radio signals at the radio receiver 19, 20, so that the quadruple MIMO system described with reference to FIG. 3 is doubled into an octuple MIMO system, without any necessary effort being required in this respect with regard to the filtering required.

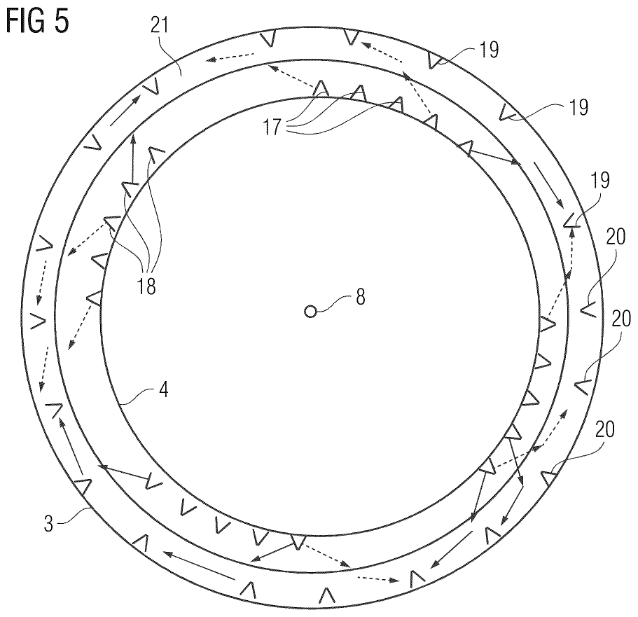

The following explains a third variant with reference to FIG. 5. FIG. 5 shows the same view of the imaging device 2 as that shown in FIG. 2 to FIG. 4. Apart from the aspects explained below, the explanations in the context of FIG. 4 apply equally to the exemplary embodiment explained with reference to FIG. 5. For the sake of clarity, FIG. 5 only depicts some of the arrows indicating the transmission of the radio signals.

In contrast to the above-described embodiments, a signal conductor 21 is provided in the third embodiment, wherein the transmission path of the radio signals from the radio transmitters 17, 18 to the radio receivers 19, 20 runs partly through the signal conductor 21. The signal conductor 21 is circular and ring-shaped and is attached to the inner circumference of the receiving portion of the stationary portion 3. The radio signals impinge laterally on the radial inner side of the signal conductor 21, are coupled into it and then propagate along a longitudinal direction or direction of extension of the signal conductor 21. The radio receivers 19, 20 are arranged within the signal conductor 21, i.e. they are embedded in its material.

In relation to FIG. 5, the radio signals generated by the radio transmitters 17 in the forward-directed radio transmitter set run clockwise through the signal conductor 21 and are received by one of the radio receivers 20 in the backward-directed radio receiver set. The radio signals generated by the radio transmitters 18 in the backward-directed radio transmitter set run counterclockwise through the signal conductor 21 and are received by one of the radio receivers 19 in the forward-directed radio receiver set.

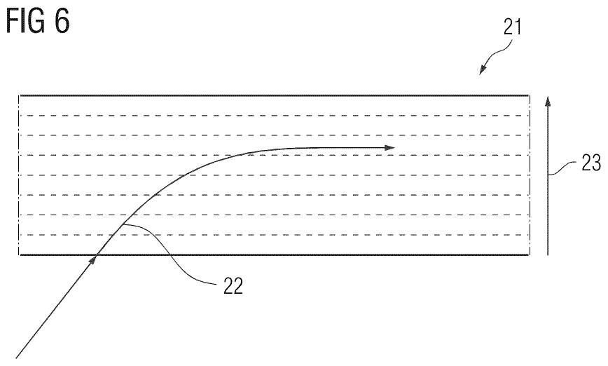

FIG. 6 shows a detailed view of a portion of the signal conductor 21, wherein the transmission path of one of the radio signals is indicated by the arrow 22. The direction pointing radially outward from the system axis 8 is indicated via the arrow 23. The signal conductor 21 consists of a dielectric material, namely a plastic such as, for example, polystyrene, polyethylene, polypropylene and/or polytetrafluorethylene. Herein, it is provided that this material or the signal conductor 21 has a permittivity that changes transversely to the direction of extension of the signal conductor 21. The direction of extension is perpendicular to the direction indicated by the arrow 23. In FIG. 6, the permittivity isolines are shown as dashed lines.

The permittivity that changes perpendicularly to the direction of extension of the signal conductor 21 leads to a continuous refraction of the electromagnetic wave representing the respective radio signal. A direction of propagation of the radio signal that initially runs at an angle to the direction of extension is deflected in the direction of extension due to the changing permittivity until the direction of propagation corresponds to the direction of extension. From this moment, the respective radio signal is guided along the direction of extension until it reaches the next radio receiver 19, 20 arranged in the signal conductor 21.

The present change in the permittivity of the signal conductor 21 is realized by the fact that its material has a changing geometric honeycomb-shaped structure. The size of a unit cell or elementary cells of this cyclically repeating pattern changes along the transverse direction of the signal conductor 21, i.e. along the direction indicated by the arrow 23. To generate this structure, the signal conductor 21 is produced via a 3D printer. Specifically, it is provided that frequencies of, for example, 60 GHz are used with regard to the radio signals, wherein the size of the unit cell or elementary cell of the honeycomb structure corresponds to a maximum of one tenth of the associated wavelength so that a continuous and therefore jump-free change in permittivity is realized.

Although the invention has been illustrated and described in greater detail by one or more example embodiments, the invention is not restricted by the disclosed examples and other variations can be derived herefrom by the person skilled in the art without departing from the scope of protection of the invention.

Independent of the grammatical term usage, individuals with male, female or other gender identities are included within the term.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuity such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible language), markup (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

1. A method for transmitting data from a rotating portion of a medical imaging device to a stationary portion of the medical imaging device, the method comprising:
transmitting the data via radio transmitters on the rotating portion and radio receivers on the stationary portion, wherein
each of a plurality of separate transmission pairs is formed, each of the plurality of separate transmission pairs includes one of the radio transmitters and one of the radio receivers, and
radio signals are transmitted from the radio transmitter to the radio receiver in the transmission pairs and a main radiation direction of the radio transmitter relating to the radio signals is tracked to the direction of the radio receiver.

2. The method of claim 1, further comprising at least one of:
specifying the transmission pairs at a start of the transmitting; or
updating the transmission pair during the transmitting in that the radio transmitter of the respective transmission pair is assigned the radio receiver which at least one of has a shortest distance to the radio transmitter or is connectable to the respective radio transmitter via a direct line of sight.

3. The method of claim 2, further comprising:
ascertaining at least one of at least one quality parameter relating to a quality of the transmission of the radio signals in the respective transmission pair or at least one rotation parameter relating to a rotation of the rotating portion, wherein the updating is based on the at least one of the at least one quality parameter or the at least one rotation parameter.

4. The method of claim 1, wherein during a complete rotation of the rotating portion, each of the radio transmitters forms one of the transmission pairs with each of the radio receivers.

5. A method for transmitting data from a rotating portion of a medical imaging device to a stationary portion of the medical imaging device, the method comprising:
transmitting the data via radio transmitters on the rotating portion and radio receivers on the stationary portion, wherein
each of a plurality of separate transmission pairs is formed, each of the plurality of separate transmission pairs includes one of the radio transmitters and one of the radio receivers, and
radio signals are transmitted from the radio transmitter to the radio receiver in the transmission pairs;
for each transmission pair, ascertaining at least one item of assignment information for the radio signals received via the radio receiver, the at least one item of assignment information describing the radio transmitter generated the radio signals; and
filtering out radio data received by the radio receiver and not generated by the radio transmitter of the transmission pair using the least one item of assignment information.

6. The method of claim 5, wherein the at least one item of assignment information is
distance information relating to a distance between the respective radio receiver and the radio transmitter via which the respective radio signal was generated,
polarization information relating to the polarization of the respective radio signal, or
frequency information relating to the frequency or a frequency band of the respective radio signal.

7. The method of claim 6, wherein the at least one item of assignment information is the frequency information, the method further comprising:
assigning the radio transmitters to one of a plurality of radio transmitter groups, wherein
the radio transmitters in one of the radio transmitter groups generate radio signals in the same frequency range, and
radio transmitters in different radio transmitter groups generate radio signals in different frequency ranges.

8. The method of claim 5, wherein the radio transmitters have at least one main radiation direction of the radio signals which is fixed with respect to the rotating portion.

9. The method of claim 8, wherein the main radiation direction of at least one of the radio transmitters and a main receiving direction of at least one of the radio receivers relating to a spatial direction that is sensitive with respect to the reception of the radio signals point at least one of along or against a direction of rotation relating to the rotation of the rotating portion.

10. The method of claim 9, further comprising:
assigning the radio transmitters to a forward-directed radio transmitter set or a backward-directed radio transmitter set, wherein
the radio transmitters with the forward-directed radio transmitter set have main radiation directions pointing along the direction of rotation,
the radio transmitters with the backward-directed radio transmitter set have main radiation directions that point against the direction of rotation; and
assigning the radio receivers to a forward-directed radio receiver set or a backward-directed radio receiver set, wherein
the radio receivers in the forward-directed radio receiver set have main receiving directions that point along the direction of rotation, and
the radio receivers in the backward-directed radio receiver set have main receiving directions that point against the direction of rotation.

11. A method for transmitting data from a rotating portion of a medical imaging device to a stationary portion of the medical imaging device, the method comprising:
transmitting the data via radio transmitters on the rotating portion and radio receivers on the stationary portion, wherein
radio signals transmitted from the radio transmitters to the radio receivers are coupled into at least one signal conductor leading to the radio receivers and
consisting of a dielectric material, and the signal conductor has a permittivity that changes transversely to a direction of extension.

12. The method of claim 11, wherein the signal conductor includes a plastic.

13. The method of claim 11, wherein the signal conductor has a changing geometric structure, and the changing permittivity is realized via the changing geometric structure.

14. The method of claim 13, wherein
the stationary portion has a cylindrical receiving region in which the rotating portion is arranged,
the radio transmitters are arranged along a circumference of the rotating portion,
the radio receivers are arranged along a circumference of the receiving region.

15. A medical imaging facility, comprising:
a medical imaging device including a rotating portion and a stationary portion, a plurality of radio transmitters on the rotating portion and a plurality of radio receivers on the stationary portion, wherein the radio transmitters and the radio receivers enable data transmission from the rotating portion to the stationary portion; and one of
a control facility configured to generate control signals to control the radio transmitters and the radio receivers such that a plurality of separate transmission pairs are formed, each transmission pair including one of the radio transmitters and one of the radio receivers, wherein
radio signals are transmitted from the radio transmitter to the radio receiver in the transmission pairs and a main radiation direction of the radio transmitter relating to the radio signals is tracked to the direction of the radio receiver;
a control facility configured to generate control signals to control the radio transmitters and the radio receivers in such that a plurality of separate transmission pairs are formed, each transmission pair including one of the radio transmitters and one of the radio receivers, wherein the control facility is configured to control the medical imaging facility such that
radio signals are transmitted from the radio transmitter to the radio receiver in the transmission pairs, at least one item of assignment information is ascertained for the radio signals received via the radio receivers, the at least one item of assignment information describes the radio transmitter which has generated the radio signals, and filter out radio data received by the radio receiver and not generated by the radio transmitter of the transmission pair using the least one item of assignment information; or
radio signals transmitted from the radio transmitters to the radio receivers can be coupled into at least one signal conductor leading to the radio receivers and consisting of a dielectric material, wherein the signal conductor has a permittivity that changes transversely to a direction of extension.

16. The method of claim 12, wherein the plastic is at least one of polystyrene, polyethylene, polypropylene, or polytetrafluorethylene.

17. The method of claim 13, wherein the signal conductor the changing geometric structure is a honeycomb-shaped structure.

18. The medical imaging facility of claim 15, wherein the medical imaging device is a computed tomography device.
